(19) United States
(12) Patent Application Publication
(10) Pub. No.: US 2005/0096556 A1
(43) Pub. Date: May 5, 2005

(54) Modular structure for heart beat signal wireless transmitter

(76) Inventor: Yi Mei Hsieh Chen, Taipei (TW)

Correspondence Address: STEVENS, DAVIS;MILLER & MOSHER, L.L.P., Suite 850, 1615 L Street, N.W., Washington, DC, 20036, US

(21) Appl. No.: 10/699,659

(22) Filed: Nov. 4, 2003

(51) Int. Cl. A61B 5/0402

(52) U.S. Cl. 600/509

(57) ABSTRACT

The invention relates to a modular structure for heart beat signal wireless transmitter with fastening belts made of conductive and water fast fabric capably fixed on the underwear by sewing and washed together with the underwear, and with several different types of modular body capably replaced and installed on the fastening belt respectively to form the heart beat signal wireless transmitter; so that the heart beat signal wireless transmitter of this invention could make use of the fastening belts with conductive fabric of high flexibility as electrode to enable a close contact between the electrode and the user's skin to precisely detect the actual heart beat signal of the user.

### BACKGROUND OF THE PRESENT INVENTION

1. Field of the Present Invention

The invention relates to a modular structure for heart beat signal wireless transmitter, particularly with fastening belts made of conductive and water fast fabric capably fixed on the underwear by sewing and washed together with the underwear, and with different types of modular body capably replaced and installed on the fastening belt to form a heart beat signal wireless transmitter.

2. Description of Prior Art

Shown in FIG. 1 is the conventional heart beat signal wireless transmitter which comprises a body (18) and a pair of rubber fastening belt (19). Attached on the underside side of the rubber fastening belts are conductive sheets which are used as two electrodes and are connected separately to the positive and negative pole of the signal transmitter on the PC board inside the body (18). With the rubber fastening belt (19) the heart beat wireless transmitter (10) can be closely bound to the user's body, so that the two conductive sheets on the underside side of the rubber fastening belts can be brought into direct contact with skin to form an electrical circuit with the PC board, therefore the ECG or actual heart beat signal detected by the conductive sheets assembly can be emitted by the signal transmitter on the PC board by way of wireless emitting, and received by a receiver which is remotely operated together with the heart beat signal wireless transmitter (10) to form a complete system for detecting and emitting the heart beat signal of the user.

However, the body (18) of conventional heart beat signal wireless transmitter (10) does not adopt modular design. The body (18) and the rubber fastening belts (19) are constructed into an integral unit which can not be separated from each other, or even though the body (10) can be separated from the rubber fastening belts (19), but only specific type of body (10) can be used together with the same type of rubber fastening belt (19). The body (10) with different type of structure can not be installed on and used together with the same rubber fastening belt (19).

Moreover, since the rubber fastening belt (19) of the conventional heart beat signal wireless transmitter (10) is made of soft rubber material which must carry conductive sheets for being used as the electrodes, therefore when the fastening belts are fixed to the clothes by sewing, and washed together with the clothes, it will cause problem of deformation or functional failure of the conductive sheets which are unable to be used. Therefore, owing to this drawback the modular design idea can not be adopted by the conventional heart beat signal wireless transmitter unless the limitation due to the aforesaid problem is overcome.

### SUMMARY OF THE PRESENT INVENTION

The major purpose of the present invention is to provide a heart beat signal wireless transmitter having new structure which comprises a fastening belt made of conductive fabric and non-conductive fabric, and the conductive fabric is used as electrode to supersede the conductive sheet on the rubber belt of prior heart beat signal wireless transmitter used that enables the heart beat signal wireless transmitter to adopt modular design, and allows the fastening belts of the same structure to be used together with the body of different structure to form the heart beat signal wireless transmitter of the invention.

Another purpose of the invention is to provide a modular design structure of the heart beat signal wireless transmitter with fastening belt made of conductive and water fast fabric which can be fixed on the underwear by sewing, and washed together with the underwear, and the body module with different structure can be mounted on and used together with the fastening belt to form the hear beat signal wireless transmitter of the invention.

The further purpose of the invention is to provide a heart beat signal wireless transmitter having new design of structure which uses the conductive fabric of high flexibility as electrode to enable a close contact between the electrode and the user's skin to precisely detect the actual heart beat signal of the user.

### BRIEF DESCRIPTION OF THE DRAWING FIGURES

FIG. 1 is the schematic drawing of the conventional heart beat signal wireless transmitter (10).

FIG. 2 is the schematic drawing of the heart beat signal wireless transmitter (20) constructed by employing the first type modular structure of the body (30) of the invention.

FIG. 3 the Schematic drawing of the fastening belt (40) of the heart beat signal wireless transmitter (20) showing the laminated structure of the fastening belt.

FIG. 4 is the disassembly drawing of the heart beat signal wireless transmitter (20) shown in FIG. 2.

FIG. 5 is the cross-sectional drawing along the cross-section line 5-5 of the heart beat signal wireless transmitter (20) shown in FIG. 2.

FIG. 6 is the schematic drawing which shows that the fastening belt (40) of the invention is made of conductive fabric, and is fastened on the underwear (15) by sewing.

FIG. 7 and FIG. 8 are the schematic drawings of the heart beat signal wireless transmitter (20) of the invention which show the modular structure of the body (30) and the fastening belt (40) fixed on the underwear (15) by sewing.

FIG. 9 is the schematic drawing of the heart beat signal wireless transmitter (20) of the invention constructed by employing the second type modular structure of the body (30).

FIG. 10 is the disassembly drawing of the heart beat signal wireless transmitter (20) shown in FIG. 9.

FIG. 11 is the cross-sectional drawing along the cross-section line 11-11 of the heart beat signal wireless transmitter (20) shown in FIG. 9.

FIG. 12 is the heart beat signal wireless transmitter (20) of the invention constructed by employing the third type modular structure of the body (30).

FIG. 13 is the disassembly drawing of the heart beat signal wireless transmitter (20) shown in FIG. 12.

FIG. 14 is the cross-sectional drawing along the cross-section line 14-14 of the heart beat signal wireless transmitter shown in FIG. 12.

FIG. 15 is the schematic drawing of the heart beat signal wireless transmitter (20) of the invention constructed by employing the forth type modular structure of the body (30).

FIG. 16 is the disassembly drawing of the heart beat signal wireless transmitter (20) shown in FIG. 15.

FIG. 17 is the cross-sectional drawing along the cross-section line 17-17 of the heart beat signal wireless transmitter (20) shown in FIG. 15.

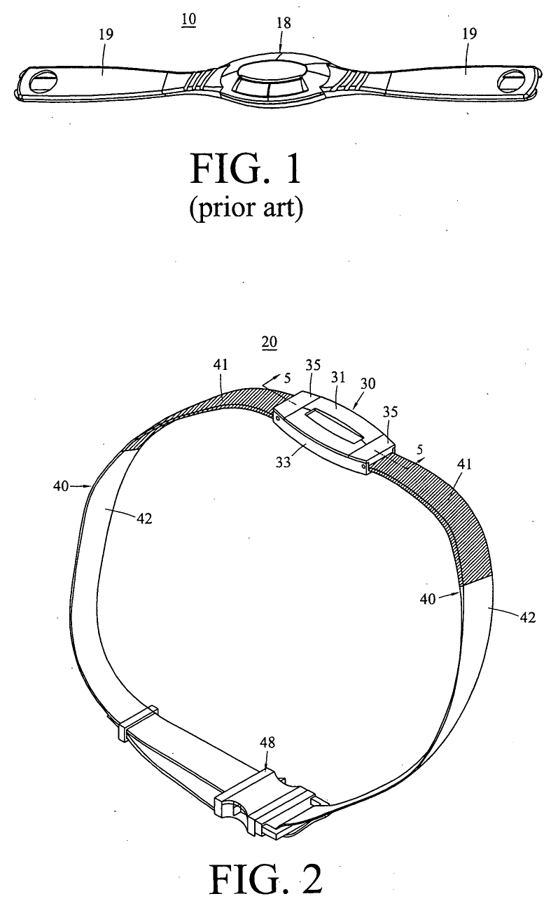

### DETAILED DESCRITION OF THE PREFERRED EMBODIMENTS

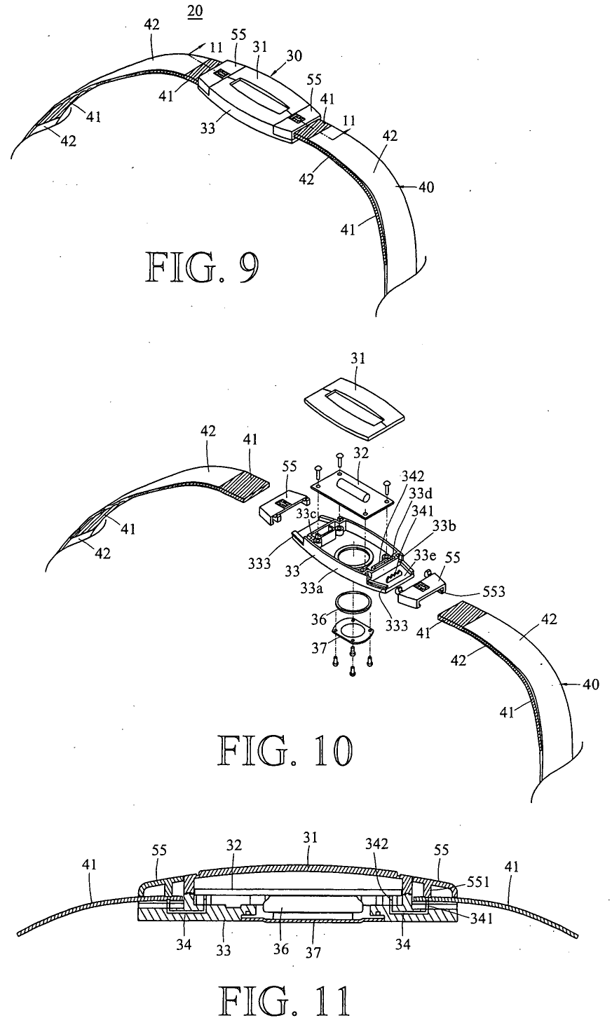

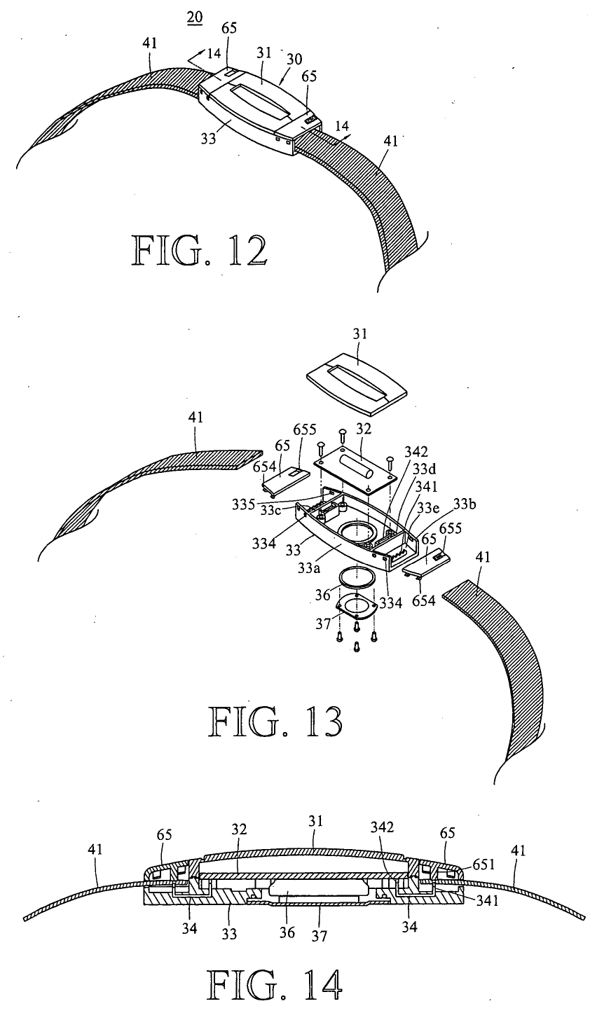

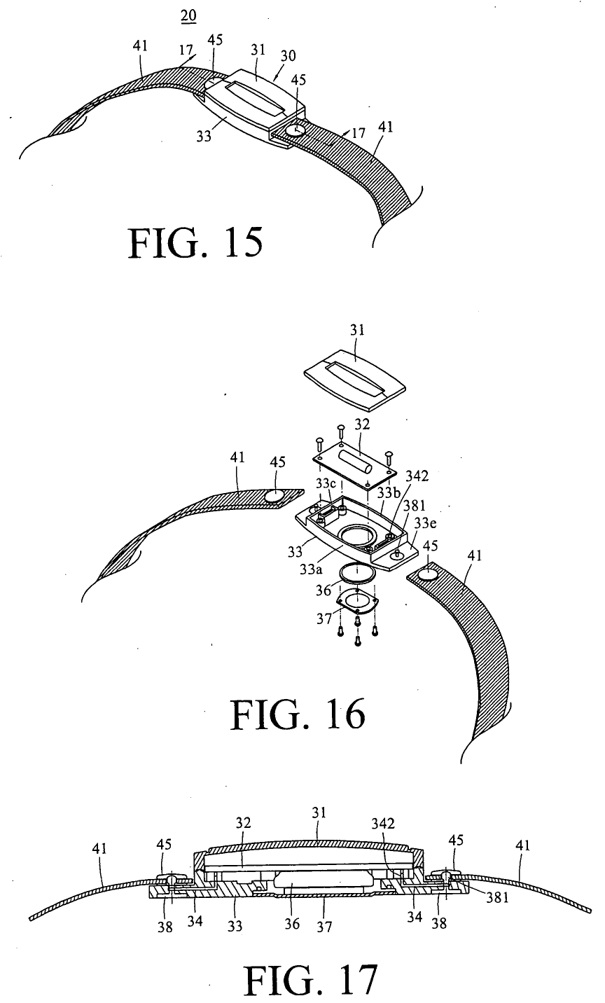

Now please see FIG. 2, FIG. 9, FIG. 12 and FIG. 15 the heart beat signal wireless transmitter (20) of the invention comprises a body (30) and a pair of detachable fastening belt (40) wherein the body (30) of the invention has four different types of modular structure all of which can be assembled and used together with the same detachable fastening belt (40) to form the heart beat signal wireless transmitter (20).

Besides, the fastening belt (40) of the invention is made of the fabric with multiple-material composite structure constructed by joining together the conductive fabric (41) and the non-conductive fabric (42), both have water fast property as shown in FIG. 2, or made of the laminated fabric formed by lamination of the conductive fabric (41) and the non-conductive fabric (42).

The said conductive fabric (41) of the fastening belt (40) is made by employing one of the conductive materials including intrinsically conductive polymer, compounds with conductive fiber and electronic fabric that enables the fabric to possess the property of good conductivity and water fast.

Therefore, the fastening belt (40) made of conductive fabric is one of the features of the heart beat signal wireless transmitter (20) of the present invention, and can be arranged into two different types of use. Shown in FIG. 2 is the first type of use which provides buckle connecting device (48) on the end of each fastening belt (40) for tiding the heart beat signal wireless transmitter (20) on the user's body with the desired body (30) having the specific modular structure installed on the fastening belt (40). Particularly, since the material of the fastening belt is made of fabric which has good flexibility to enable a close contact between the belt and the skin of the user that brings the conductive fabric (41) into close contact with user's skin to enable a precise detecting of the heart beat signal of user.

The second type of use of the fastening belt is shown in FIG. 6 and FIG. 8. Part of the pair of fastening belt (40) made of water fast fabric are fixed on the underwear (15) or bra (16) by sewing to form an integral unit, i.e. the non-conductive fabric (42) of the fastening belt (40) and part of the conductive fabric (41) for being used as electrode are fixed on the inner side of the underwear (15) while the other part of the two conductive fabrics (41) are exposed on the outer side of the underwear (15), with front end connected to the body (30) of the invention, and the conductive fabric (41) fixed on the inner side of the underwear by sewing can form a close contact with user's skin, therefore, simply by installing the desired type of the body (30) of the invention a heart beat signal wireless transmitter (20) for detecting heart beat can be formed, and the underwear (15) carrying the fastening belt (40) can be washed for reuse after the body (30) is detached from the fastening belt (40).

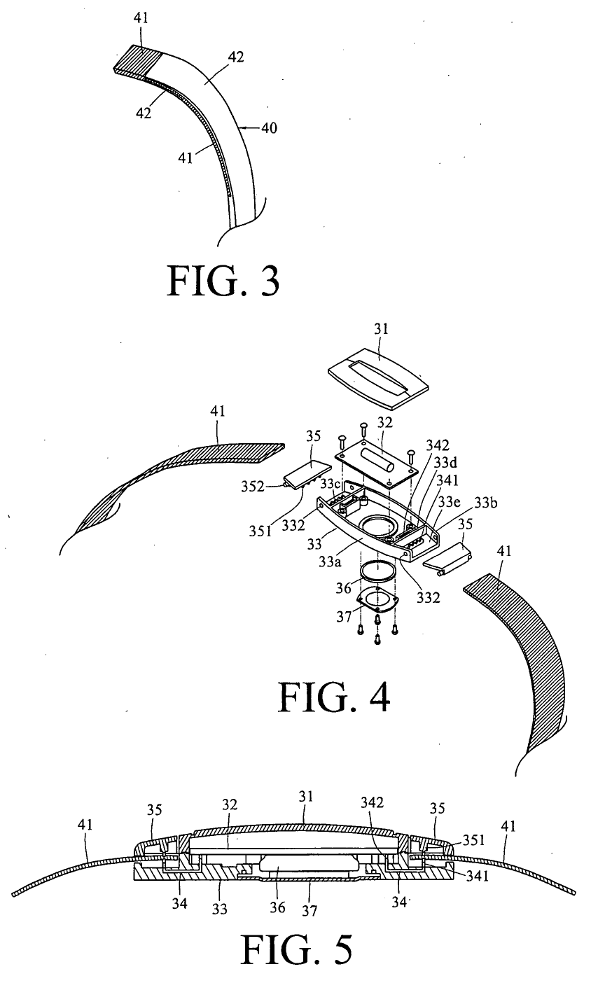

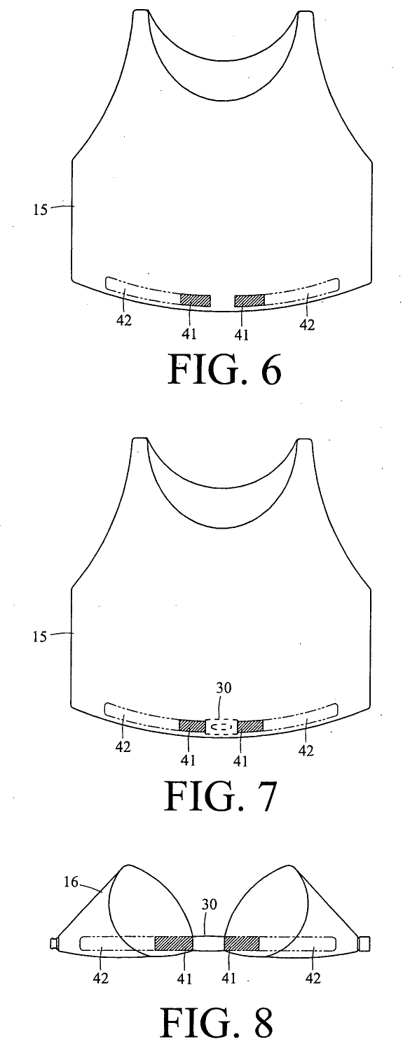

The body (30) described in the present invention has four different types of structure, the first type of structure is shown in the drawings from FIG. 2 through FIG. 5 which comprises an upper cover (31), a PC board (32) which carries the signal transmitter, a seat (33), a clamping plate (35), a battery (36) and a battery compartment cover (37).

In the central position of the seat (33) is a compartment for installing the PC board (32), and two openings are formed on the left and right side of the seat (33) for installing the clamping plate (35), i.e., as shown in FIG. 5, the seat (33) is formed by a front wall (33a), a rear wall (33b), a left partition board (33c), a right partition board (33d) and a bottom plate (33e) to construct a structure having a compartment in central position and opening on left and right side. When the PC board (32) carrying signal transmitter is installed in the compartment on the seat (33), the upper cover (31) is installed on the seat (33) to close the compartment. Meanwhile, the battery (36) is served as the electric power source of the PC board (32), and is fixed inside the battery compartment by the battery compartment cover (37) which is mounted on the bottom plate (33e) of the seat (33) to supply electric power to the PC board (32).

Installed on both of the left and right side of the bottom plate (33e) of the seat (33) are two U shaped contact inserts (34). The horizontal part of the contact insert (34) is inserted into the space between the partition board and the bottom plate (33e) with one vertical end exposed on the outer side of the compartment on the seat for mounting the PC board (32) having its top side formed into saw-toothed grip piece (341), and another vertical end is exposed inside the compartment of the seat (33) for mounting the PC board (32) to form a contact pin to get into close contact with either positive or negative pole on the underside of the PC board (32) forming an electric circuit, meanwhile, pivot holes (332) are provided on both sides of the front wall (33a) and the rear wall (33b) for mounting the pivot (352) of the clamping plate (35).

The shape and size of the clamping plate (35) is arranged to match the opening on the left and right side of the seat (33), and formed on the underside side of the clamping plate is a saw-toothed grip piece (351) which when the clamping plate is mounted on the front and rear wall of the seat (33) by installing its pivots into the pivot holes (332) can form a clamp assembly with movable clamping plate able to be lifted up and pushed down around the pivot center.

When the clamping plate (35) is lifted up, a gap or space is formed between the clamping plate (35) and seat (33) of the body sufficiently for holding the conductive fabric (41) of the fastening belt (40) in the openings on both sides of the seat (33), then push the clamping plate (35) down to cover the openings on both sides of the seat (33), and have the saw-toothed grip piece (351) of the clamping plate (35) engaged with the saw-toothed grip piece (341) of seat (33) to securely grip the conductive fabric (41) and form an satisfied electric connection between the saw-toothed grip piece (341) and the conductive fabric (41) of the fastening belt (40) which enables the conductive fabric (41) of the fastening belt (40) to form the positive or negative electrode of the PC board (32) that forms a heart beat signal wireless transmitter (20) for detecting the heart beat signal of the user.

Again, when the clamping plate (35) of the body (30) is lifted up, the engaging condition between the clamping plate (35) and the seat (33) is immediately released to let the conductive fabric (41) of the fastening belts be detached from the body (30), or another type of body (30) of different structure can be connected to the fastening belt (40) for replacement to form another type of assembly of the heart beat signal wireless transmitter (20).

The second type of modular structure of the body (30) of the invention is shown in the drawings from FIG. 9 through FIG. 11 which comprises an upper cover (31), a PC board (32) carrying a signal transmitter, a seat (33), a slip plate (55), a battery (36) and a battery compartment cover (37). The seat (33) of the second type structure of the body (30) is the same as the seat (33) of the first type structure of the body (30) except the outer surface of both sides of the front and rear wall have mounting slot (333).

The shape and size of the slip plate (55) are arranged to match the opening on the left and right side of the seat (33) with a saw-toothed grip piece (551) formed on the underside of the slip plate (55) and mounting rib (553) formed on the lower end of both sides of the vertical walls of the slip plate to engage with the mounting slots (333) to connect the slip plate and the seat (33) to form a complete unit.

When the conductive fabric (41) of the fastening belt (40) is inserted into the opening on the left and right side of the seat (33) a secure connection can be formed simply by installing the slip plate (55) on the seat (33) to have the mounting rib (553) engaged with the mounting slot (333), that the conductive fabric (41) of the fastening belt (40) can be securely gripped by the saw-toothed grip piece (551) of the slip plate (55) and the saw-toothed grip piece (341) of the seat (33), and conversely when the slip plate (55) is pushed back and detached from the seat (33), the conductive fabric (41) of the fastening belt (40) can be freely detached from the body (30), or the body (30) can be replaced with another body with different type of structure to form a heart beat signal wireless transmitter (20) for detecting the heart beat signal.

The third type of modular structure of the body (30) of the invention is shown in the drawings from FIG. 12 through FIG. 14 which comprises an upper cover (31), a PC board (32) carrying signal transmitter, a seat (33), a press-in cover (65), a battery (36) and a battery compartment cover (37).

The seat (33) of the third type of modular structure of body (30) is generally the same as that of the first type structure of the body (30). The press-in cover (65) has a saw-toothed grip piece (651) on the underside, and has mounting pins and spring snap piece (655) formed on its two sides with respect to the pin holes (334) and snap hole (335) on the seat (33); Therefore, when mounting the press-in cover (65) on the seat (33), the mounting pins (654) of the press-in cover (65) are inserted into the pin holes (334) on the seat (33), than push the press-in cover against the opening on both sides of the seat (33) to have the spring snap piece (655) on the press-in cover (65) snapped into the snap hole (335) to form a complete unit with the seat (33). With this when the conductive fabric (41) of the fastening belt (40) is inserted into the opening on both sides of the seat (33), and the press-in cover (65) is in normal mounting condition the saw-toothed grip piece (651) on the press-in cover (65) can engage with the saw-toothed grip piece (341) on the seat (33) that can securely grip the conductive fabric (41) of the fastening belt (40); conversely, when the press-in cover (65) and the seat (33) are separated, the conductive fabric (41) of the fastening belt (40) can be freely detached from the body (30) it originally connected to, or another body (30) of different structure can be connected to the fastening belt to form a heart beat signal wireless transmitter (20) for detecting the heart beat signal of the user.

The fourth type modular structure of the body (30) is shown in the drawings from FIG. 15 through FIG. 17 which comprises a upper cover (31), a PC board (32) carrying signal transmitter, a seat (33), a battery (36), a fixing cap (35), and a battery compartment cover (37). The fastening belt (40) used for this type of body (30) has a female connecting hole (45) formed on the conductive fabric (41).

The seat (33) of the fourth type structure of the body (30) is generally the same as that of the first type structure of the body (30) except the U shaped contact insert (34) used on this type of body (30) has no saw-toothed grip piece (341) as described above which was replaced by a male connecting piece (38) having a connecting head (381) installed on the left and right side of the seat (33) forming an electrical connection with the contacting insert (34). Therefore, when the female connecting hole (45) on the conductive fabric (41) of the fastening belt (40) engages with the male connecting head (381), and fixed by the fixing cap (35), the conductive fabric (41) of the fastening belt (40) forms the positive or negative electrode of the PC board (32) that constructs a complete system of heart beat signal wireless transmitter (20) for detecting the heart beat signal of the user. Conversely when the female connecting hole (45) disengages with the male connecting head (381), the conductive fabric (41) of the fastening belt (40) can be freely detached from the body (30).

1. A heart beat signal wireless transmitter comprising a body which constructs a modular structure wherein the said body comprises a PC board carrying a signal transmitter and a pair of detachable fastening belt connected to the body separately through the claming means provided on both sides of the body, and the said pair of fastening belt is made of fabric material of which the front part of each fastening belt is made of conductive fabric for forming the electrical connection with the PC beard inside the body when the pair of fastening belt and the body are connected to each other through the clamping means on both sides of the body.

2. The heart beat signal wireless transmitter as defined in claim 1, wherein the detachable fastening belt is made of conductive fabric and non-conductive fabric.

3. The heart beat signal wireless transmitter as defined in claim 1, wherein the conductive fabric of the fastening belt is made of one of the materials of intrinsically conductive polymer, compounds with conductive fiber and electronic fabric.

4. The heart beat signal wireless transmitter as defined in claim 2, wherein the conductive fabric of the fastening belt is made of one of the materials of intrinsically conductive polymer, compounds with conductive fiber and electronic fabric.

5. The heart beat signal wireless transmitter as defined in claim 1, wherein the said pair of detachable fastening belt is fixed on the underwear by sewing.

6. The heart beat signal wireless transmitter as defined in claim 2, wherein the said pair of detachable fastening belt is fixed on the underwear by sewing.

7. The heart beat signal wireless transmitter as defined in claim 1, wherein the said pair of fastening belt is fixed on the bra by sewing.

8. The heart beat signal wireless transmitter as defined in claim 2, wherein the said pair of fastening belt is fixed on the bra by sewing.

9. The heart beat signal wireless transmitter as defined in claim 1, wherein the end portion of the said pair of fastening belt has a buckle assembly for buckling the fastening belt.

10. The heart beat signal wireless transmitter as defined in claim 2, wherein the end portion of the said pair of fastening belt has a buckle assembly for buckling the fastening belt.

11. The heart beat signal wireless transmitter as defined in claim 1, wherein a clamping plate having saw-toothed grip piece on the underside is pivotally installed on both sides of the said body, and can be lifted up and pressed down around the center of the pivot.

12. The heart beat signal wireless transmitter as defined in claim 2, wherein a clamping plate having saw-toothed grip piece on the underside is pivotally installed on both sides of the said body, and can be lifted up and pressed down around the center of the pivot.

13. The heart beat signal wireless transmitter as defined in claim 1, wherein a slip plate having saw-toothed grip piece on the underside is installed on both sides of the said body through tenon-slot structure.

14. The heart beat signal wireless transmitter as defined in claim 2, wherein a slip plate having saw-toothed grip piece on the underside is installed on both sides of the said body through tenon-slot structure.

15. The heart beat signal wireless transmitter as defined in claim 1, wherein a press-in cover equipped with a spring snap piece, having saw-toothed grip piece on underside is installed on both sides of said body.

16. The heart beat signal wireless transmitter as defined in claim 2, wherein a press-in cover equipped with a spring snap piece, having saw-toothed grip piece on underside is installed on both sides of said body.

17. The heart beat signal wireless transmitter as defined in claim 1, wherein a female connecting hole is provided on the conductive fabric of the pair of fastening belt, and a male connecting head is provided on both sides of said body.

18. The heart beat signal wireless transmitter as defined in claim 2, wherein a female connecting hole is provided on the conductive fabric of the pair of fastening belt, and a male connecting head is provided on both sides of said body.
